(19) United States
(12) Patent Application Publication
(10) Pub. No.: US 2015/0112304 A1
(43) Pub. Date: Apr. 23, 2015

(54) APPARATUS HAVING A SELECTIVELY CURVED DISTAL END AND METHODS FOR USE

(71) Applicant: INVATEC S.p.A, Roncadelle (IT)

(72) Inventor: Claudio Silvestro, Roncadelle (IT)

(73) Assignee: INVATEC S.p.A, Roncadelle (IT)

(21) Appl. No.: 14/058,444

(22) Filed: Oct. 21, 2013

(51) Int. Cl. A61M 25/00

(52) U.S. Cl. CPC A61M 25/0043
USPC 604/508; 604/170.03

(57) ABSTRACT

An apparatus having a distal end that may be selectively bent in situ. The apparatus has a curved configuration and a substantially straightened configuration. First and second tubular components of the apparatus each include a distal portion that is at an acute angle with respect to a longitudinal axis of the apparatus. The second tubular component is rotatably disposed within the first tubular component. In the curved configuration, the distal portions of the first and second tubular components are positioned to extend at substantially the same acute angle with respect to the longitudinal axis of the apparatus. In the substantially straightened configuration, the distal portions of the first and second tubular components are positioned such that the curvatures thereof counterbalance each other. The first and second tubular components may be used in an occlusion bypassing apparatus for re-entering the true lumen of a vessel after subintimally bypassing an occlusion.

### FIELD OF THE INVENTION

The invention relates generally to catheters, and in particular to apparatuses having a distal end that may be selectively bent in situ.

### BACKGROUND OF THE INVENTION

A variety of catheters for delivering a therapy and/or monitoring a physiological condition have been implanted or proposed for implantation in patients. Catheters may deliver therapy to, and/or monitor conditions associated with, the heart, muscle, nerve, brain, stomach or other organs or tissue. Many catheters are tracked through the vasculature to locate a therapeutic or diagnostic portion of the catheter at a target site. Such catheters must have flexibility to navigate the twists and turns of the vasculature, sufficient stiffness in the proximal portion thereof to be pushed through the vasculature alone or over a guidewire or through a lumen, and the capability of orienting a distal portion thereof in alignment with an anatomical feature at the target site so that a diagnostic or therapeutic procedure can be completed. In general terms, the catheter body must also resist kinking and be capable of being advanced through access pathways that twist and turn, sometimes abruptly at acute angles.

The distal portions of catheters frequently need to be selectively curved or bent and straightened again while being advanced within the patient to steer the catheter distal end into a desired body lumen or chamber. For example, it may be necessary to direct the catheter distal end through tortuous anatomies and/or into a branch at a vessel bifurcation. In addition, some procedures require high accuracy in guidewire orientation. For example, often patient's arteries are irregularly shaped, highly tortuous and very narrow. The tortuous configuration of the arteries may present difficulties to a clinician in advancement of a catheter to a treatment site. In addition, in some instances, the extent to which a lumen is narrowed at the treatment site is so severe that the lumen is completely or nearly completely obstructed, which may be described as a total occlusion. Total or near-total occlusions in arteries can prevent all or nearly all of the blood flow through the affected arteries. If the total or near total occlusion has been established for a long period of time, the lesion may be referred to as a chronic total occlusion or CTO. Chronic total occlusions can occur in coronary as well as peripheral arteries. Chronic total occlusions are often characterized by extensive plaque formation and typically include a fibrous cap surrounding softer plaque material. This fibrous cap may present a surface that is difficult to penetrate with a conventional medical guidewire.

A number of devices have been developed and/or used for the percutaneous interventional treatment of CTOs, such as stiffer guidewires, low-profile balloons, laser light emitting wires, atherectomy devices, drills, drug eluting stents, and re-entry catheters. The factor that is most determinative of whether the physician can successfully recanalize a CTO is the physician's ability to advance a suitable guidewire from a position within the true lumen of the artery proximal to the CTO lesion, across the CTO lesion, i.e., either through the lesion or around it, and then back into the true lumen of the artery at a location distal to the CTO lesion.

In some cases, such as where the artery is totally occluded by hard, calcified atherosclerotic plaque, the guidewire may tend to deviate to one side and penetrate through the intima of the artery, thereby creating a neo-lumen called a “subintimal tract,” i.e., a penetration tract formed within the wall of the artery between the intima and adventitia. In these cases, the distal end of the guidewire may be advanced to a position distal to the lesion but remains trapped within the subintimal tract. In such instances, it is then necessary to direct or steer the guidewire from the subintimal tract back into the true lumen of the artery at a location distal to the CTO lesion. The process of manipulating the guidewire to reenter the artery lumen is often difficult and various solutions have been proposed utilizing means for handling such a reentry operation.

As well a number of catheter-based devices have been heretofore suggested for redirecting subintimally placed guidewires or other medical devices back into the true lumen of the artery. Included among these are a variety of catheters having laterally deployable cannulae, i.e., hollow needles. For example, some catheter systems utilize a penetrator or needle that exits through a side exit port of the catheter to puncture the intimal layer distal of the CTO to re-enter the true lumen of the vessel. A second guidewire is then passed through the laterally deployed needle and is advanced into the true lumen of the artery. However, a need in the art still exists for other medical devices or systems that consistently and reliably direct guidewires or other devices tracked within the subintimal space of a vessel back into the true lumen of the vessel for the treatment of a CTO. Further, a need in the art still generally exists for improved apparatuses and methods for navigating through or within a patient's anatomy.

### BRIEF SUMMARY OF THE INVENTION

Embodiments hereof are directed to apparatuses for use within a vasculature of a patient. In an embodiment, the apparatus includes a first tubular component and a second tubular component. The first tubular component has a distal portion that is at an acute angle with respect to a longitudinal axis of the apparatus. The second tubular component is rotatably disposed within the first tubular component and sized to be rotatable relative thereto, and also has a distal portion that is at an acute angle with respect to the longitudinal axis of the apparatus. The apparatus has a curved configuration and a substantially straightened configuration. In the curved configuration of the apparatus, the distal portions of the first and second tubular components are positioned to extend at substantially the same acute angle with respect to the longitudinal axis of the apparatus. In the substantially straightened configuration of the apparatus, the distal portions of the first and second tubular components are positioned such that the curvatures of the first and second tubular components counterbalance each other.

In another embodiment, the apparatus includes a first tubular component and a second tubular component. The first tubular component defines a lumen from a proximal end to a distal end thereof and has an elbow beyond which a distal portion of the first tubular component is at an acute angle with respect to a longitudinal axis of the apparatus. The second tubular component is disposed within the lumen of the first tubular component and sized to be rotatable relative thereto. The second tubular component also has an elbow beyond which a distal portion of the second tubular component is at an acute angle with respect to the longitudinal axis of the apparatus. The apparatus has a first configuration and a second configuration. In the first configuration of the apparatus, the elbows of the first and second tubular components are aligned with each other such that the distal portions of the first and second tubular components are bent at substantially the same acute angle with respect to the longitudinal axis of the apparatus. In the second configuration of the apparatus, the elbows of the first and second tubular components are oriented in opposite directions from each other such that the distal portions of the first and second tubular components are straightened to substantially extend along the longitudinal axis of the apparatus.

Embodiments hereof also relate to methods of orienting in situ a distal end of an apparatus. The apparatus is percutaneously advanced through a vasculature to a target location. The apparatus includes a first tubular component and a second tubular component rotatably disposed within the first tubular component. Each of the first and second tubular components has a distal portion that is at an acute angle with respect to the longitudinal axis of the apparatus. During the step of advancing the apparatus through a vasculature, the apparatus is in a substantially straightened configuration in which the distal portions of the first and second tubular components are positioned such that the curvatures of the first and second tubular components counterbalance each other. Then, at the target location, the first tubular component and the second tubular component are rotated relative to each other to configure the apparatus into a curved configuration. In the curved configuration, the distal portions of the first and second tubular components are aligned to extend at substantially the same acute angle with respect to the longitudinal axis of the apparatus.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a bendable subassembly for use in a catheter apparatus, wherein the bendable subassembly has a distal end that may be selectively bent or curved in situ and is in a substantially straightened or delivery configuration.

FIG. 2 is a side view of the bendable subassembly of FIG. 1, wherein the bendable subassembly is in a curved or bent configuration.

FIG. 3 is a side view of a first tubular component of the bendable subassembly of FIG. 1 in accordance with an embodiment hereof.

FIG. 3A is a cross-sectional view of the first tubular component of FIG. 3 taken along line A-A thereof.

FIG. 4 is a side view of a second tubular component of the bendable subassembly of FIG. 1 in accordance with an embodiment hereof.

FIG. 4A is a cross-sectional view of the second tubular component of FIG. 4 taken along line A-A thereof.

FIG. 5 is a side view of an occlusion bypassing apparatus according to an embodiment hereof, wherein the apparatus is in a substantially straightened or delivery configuration.

FIG. 5A is a cross-sectional view of the occlusion bypassing apparatus of FIG. 5 taken along line A-A thereof.

FIG. 6 is a side view of an outer shaft component of the occlusion bypassing apparatus of FIG. 5 in accordance with an embodiment hereof.

FIG. 7 is a side view of the distal portion of the occlusion bypassing apparatus shown in FIG. 5, wherein the apparatus is in a substantially straightened or delivery configuration and a guidewire extends there-through.

FIG. 8 is a side view of the distal portion of the occlusion bypassing apparatus shown in FIG. 1, wherein the apparatus is in a curved or bent configuration and a needle extends there-through.

FIG. 9 is a side view of the needle of FIG. 8, wherein the needle is removed from the occlusion bypassing system for illustrative purposes.

FIG. 9A is a cross-sectional view of the needle of FIG. 9 taken along line A-A thereof.

FIG. 10 is a diagram of an artery showing the three layers of tissue that comprise the artery wall.

FIGS. 11-21 illustrate the steps of utilizing the occlusion bypassing apparatus of FIG. 1 to bypass a chronic total occlusion according to an embodiment hereof.

FIGS. 22-27 illustrate the steps of utilizing the occlusion bypassing apparatus of FIG. 1 to bypass a chronic total occlusion according to another embodiment hereof.

FIG. 28 illustrates utilization of the bendable subassembly of FIG. 1 to navigate within a bifurcation according to an embodiment hereof.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. The term “shape memory” is used in the following description with reference to the tubular components hereof and is intended to convey that the structures are shaped or formed from a material that can be provided with a mechanical memory to return the structure from a straightened delivery configuration to an angled or bent configuration. Non-exhaustive exemplary materials that may be imparted with a shape memory include stainless steel, a pseudo-elastic metal such as a nickel titanium alloy or nitinol, a so-called super alloy, which may have a base metal of nickel, cobalt, chromium, or other metal, or a polymer having a shape memory such as but not limited to polyetheretherketone (PEEK). Shape memory may be imparted to a tubular or rod-like structure by thermal treatment to achieve a spring temper in stainless steel, for example, or to set a mechanical memory in a susceptible metal alloy, such as nitinol. In addition, the terms “substantially straightened” or “substantially straight” or “straightened” or “straight” are used in the following description with reference to the tubular components hereof and are intended to convey that the structure(s) extend parallel or in line with a longitudinal axis L_{A} of the apparatus or bendable subassembly of which the structure is a component.

The following detailed description is merely exemplary in nature and is not intended to limit the invention or the application and uses of the invention. Although the description of the invention is in the context of treatment of blood vessels such as smaller diameter peripheral or coronary arteries, the invention may also be used in any other body passageways where it is deemed useful. Although the description of the invention generally refers to an apparatus and method of bypassing a vessel blockage in a proximal-to-distal direction, i.e. antegrade or with the blood flow, the invention may be used equally well to bypass a vessel blockage in a distal-to-proximal direction, i.e. retrograde or against the blood flow if access is available from that direction. In other terms, the system and method described herein may be considered to bypass a vessel blockage from a near side of the blockage to a far side of the blockage or vice versa. Furthermore, there is no intention to be bound by any expressed or implied theory presented in the preceding technical field, background, brief summary or the following detailed description.

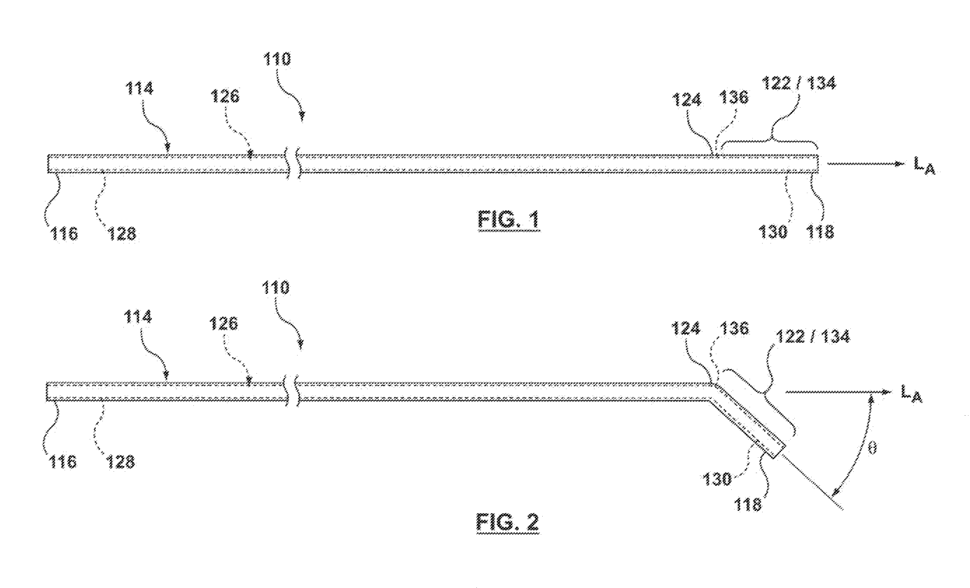

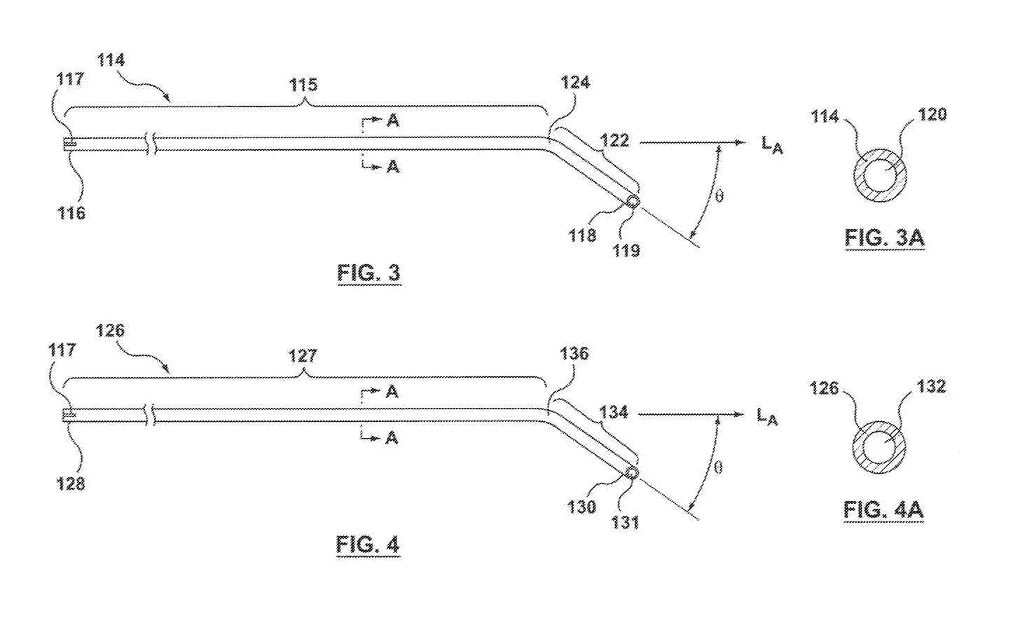

Embodiments hereof relate to catheters or similar apparatuses having a distal end that may be selectively bent or curved in situ. More particularly, with reference to FIGS. 1-4, a selectively directable or bendable subassembly 110 that may be utilized in a variety of catheters or similar apparatuses is shown. Bendable subassembly 110 includes a first or outer tubular component 114 and a second or inner tubular component 126 slidably and rotatably disposed within first tubular component 114. FIGS. 1 and 2 illustrate second tubular component 126 concentrically disposed within first tubular component 114, while FIG. 3 shows first tubular component 114 in its pre-set, shape memory curved or angled form, and FIG. 4 shows second tubular component 126 in its pre-set, shape memory curved or angled form. As shown in FIGS. 1-4, first and second tubular components 114, 126 each include a distal portion 122, 134, respectively, that is at an acute angle with respect to a longitudinal axis L_{A} of the bendable subassembly. As will be explained in more detail herein, first and second tubular components 114, 126 may be rotated relative to each other to selectively alternate or transform bendable subassembly 110 between a substantially straightened or delivery configuration that is shown in FIG. 1 and a curved or bent configuration that is shown in FIG. 2. Thus, due to relative rotation between first and second tubular components 114, 126, distal portions 122, 134 thereof are selectively bent or curved in order to orient distal openings 119, 131 thereof in situ, as will be explained in more detail herein.

As best shown in FIG. 3, which shows first tubular component 114 in its pre-set, shape memory curved or angled form, first tubular component 114 is an elongate tubular or cylindrical element having an elongated substantially straight first or proximal portion 115 and an elbow or flexion point 124 beyond which second or distal portion 122 extends, bends, or otherwise curves at an acute angle θ with respect to proximal portion 115 and the longitudinal axis L_{A} of the bendable subassembly. In embodiments hereof, angle θ may be in the range of 20° to 80°. With additional reference to FIG. 3A, which is a cross-sectional view taken along line A-A of FIG. 3, first tubular component 114 defines a continuous lumen 120 from a proximal end 116 to a distal end 118 thereof.

Second tubular component 126 is sized to be slidably and rotatably disposed within lumen 120 of first tubular component 114. As used herein, “slidably” denotes back and forth movement in a longitudinal direction while “rotatably” denotes movement or rotation about a longitudinal axis L_{A} of bendable subassembly 110. Although second tubular component 126 is smaller in diameter than first tubular component, second tubular component 126 has a structure similar to first tubular component 114 in that the second tubular component has the same overall shape and/or profile than the first tubular component. More particularly, as best shown in FIG. 4 which shows second tubular component 126 in its pre-set, shape memory curved or angled form, second tubular component 126 is an elongate tubular or cylindrical element having an elongated substantially straight first or proximal portion 127 and an elbow or flexion point 136 beyond which second or distal portion 134 extends, bends, or otherwise curves at acute angle θ with respect to proximal portion 127 and the longitudinal axis L_{A} of the bendable subassembly. The acute angles θ of first and second tubular components are equal to each other or substantially equal to each other, with “substantially” as used herein including angles within 10° of each other. In an embodiment hereof, a slight difference in the acute angles θ of first and second tubular components may advantageously allow for easy sliding or longitudinal movement between the first and second tubular components. With additional reference to FIG. 4A, which is a cross-sectional view taken along line A-A of FIG. 4, second tubular component 126 defines a continuous lumen 132 from a proximal end 128 to a distal end 130 thereof. As will be explained in more detail herein, depending upon the application thereof, lumen 132 of second tubular component 126 is sized to slidably receive an elongated component such as but not limited to a guidewire component and/or a needle component.

At least distal portions 122, 134 of first and second tubular components 114, 126, respectively, are preferably formed from a shape memory material such that a heat or thermal treatment thereof sets the shape memory of distal portions 122, 134 to curve or bend away from longitudinal axis L_{A} thereof at the acute angle θ. As previously discussed, examples of the shape memory material include but are not limited to nitinol, which utilizes the elastic properties of stress induced martensite, thermally treated stainless steel having a spring temper, or a polymer such as but not limited to polyetheretherketone (PEEK). In an embodiment, first and second tubular components 114, 126 may be elongate elements of shape memory material with a distal portion that has been shape set in an angled configuration. In another embodiment, first and second tubular components 114, 126 may be formed from more than one material, e.g. with the elongated proximal portions 115, 127 being formed of a first material not having shape memory such as stainless steel, and only angled distal portions 122, 134 being formed of a shape memory material such as nitinol.

First and second tubular components 114, 126 may be rotated relative to each other to selectively configure or transform the bendable subassembly into the substantially straightened or delivery configuration as shown in FIG. 1 and the curved or bent configuration shown in FIG. 2. Stated another way, rotation of first tubular component 114 relative to second tubular component 126, or rotation of second tubular component 126 relative to first tubular component 114, configures or transforms the bendable subassembly into the curved and straightened configurations. More particularly, in the substantially straightened or delivery configuration of bendable subassembly 110, distal portions 122, 134 of first and second tubular components 114, 126 are positioned such that the curvatures thereof counterbalance or straighten each other such that they each extend substantially parallel with respect to the longitudinal axis L_{A} of the bendable subassembly as shown in FIG. 1. Elbows 124, 136, respectively, of first and second tubular components 114, 126 are curved or oriented in opposite directions from each other such that distal portions 122, 134 of first and second tubular components 114, 126 are straightened to substantially extend along the longitudinal axis L_{A} of the bendable subassembly. When the curvatures of elbows 124, 136 are not aligned, the internal restoring forces of each shape memory tubular component, i.e., first and second tubular components 114, 126, act against each other and result in a substantially straightened bendable subassembly 110. Stated another way, the curved forms of first and second tubular components 114, 126 counterbalance, counteract, offset, or otherwise cancel out each other. As used herein, the terms counterbalance, counteract, and offset refer to the equal opposing internal restorative forces of first and second tubular components 114, 126, due to the pre-set shape memory thereof, when the elbows 124, 136 of the first and second tubular components are curved or oriented in opposite directions from each other.

In the curved or bent configuration of bendable subassembly 110, distal portions 122, 134 of first and second tubular components 114, 126 are aligned to extend at substantially the same acute angle with respect to the longitudinal axis L_{A} of the bendable subassembly as shown in FIG. 2. Elbows 124, 136, respectively, of first and second tubular components 114, 126 are aligned with or overlap each other such that the distal portions 122, 134 of first and second tubular components 114, 126 are bent or flexed at substantially the same acute angle θ with respect to the longitudinal axis L_{A} of the bendable subassembly. When elbows 124, 136 are aligned or overlap, each distal portion 122, 134 resumes its shape memory geometry by its own internal restoring forces and such that they concurrently bend, curve, or bow away from the longitudinal axis L_{A} of bendable subassembly 110. As described above, angle θ may be in the range of 20° to 80°. Bending of distal portions 122, 134 permits a user or clinician to selectively orient distal openings 119, 131 of first and second tubular components 114, 126 in situ as described in more detail herein.

In order to facilitate rotation between first tubular component 114 and second tubular component 126 to transform the bendable subassembly into the curved or straightened configuration, one or both of first tubular component 114 and second tubular component 126 may include a tag or marker 117 (shown on FIGS. 3 and 4). More particularly, marker 117 may be attached to proximal end 116 of first tubular component 114 and/or proximal end 128 of second tubular component 126. When it is desired to transform the bendable subassembly into the curved or straightened configuration, first tubular component 114 and second tubular component 126 must be rotated with respect to each other approximately 180°. “Approximately” as used herein with respect to the required degree of rotation includes relative rotation of the two tubular components between 170 degrees and 190 degrees. Marker(s) 117 assist the user in tracking the degree of rotation of the tubular components. The changed position of marker(s) 117 denotes when first and second tubular components 114, 126 have been rotated with respect to each other approximately 180 degrees, thereby indicating when the desired straight or curved configuration has been achieved.

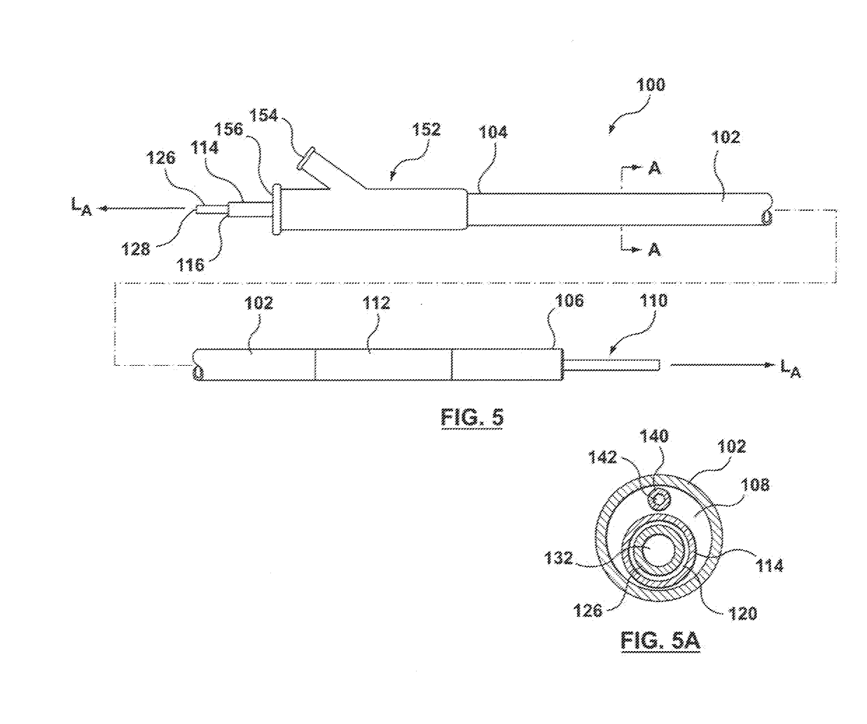

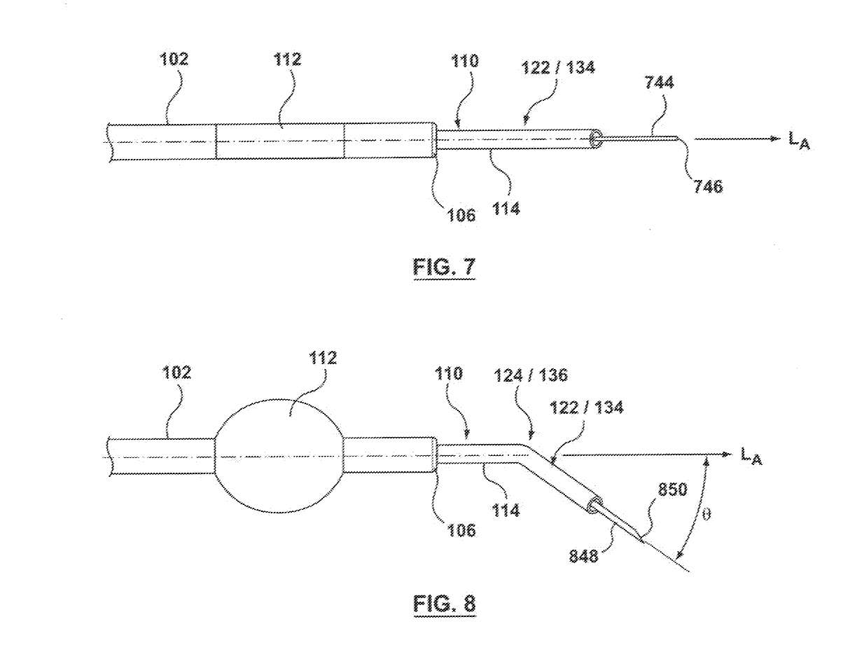

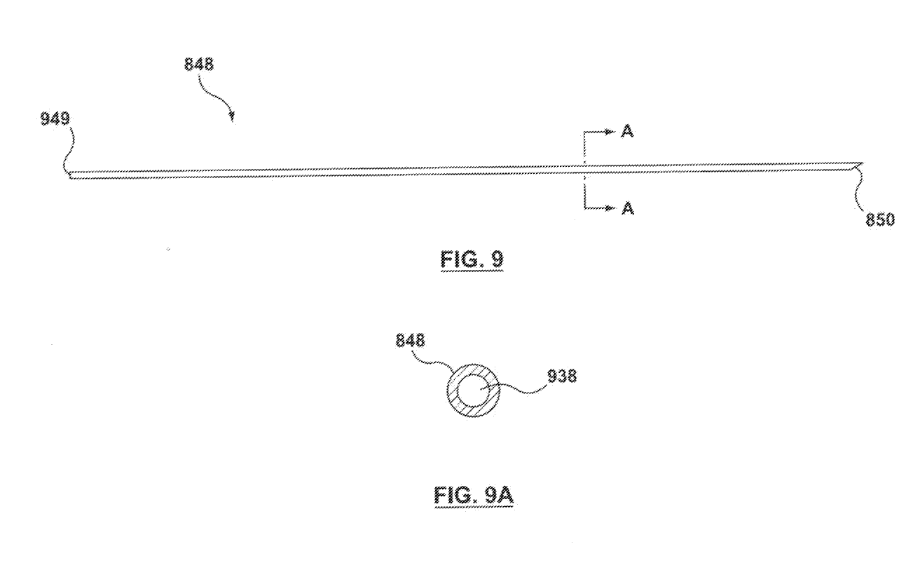

In an embodiment hereof, bendable subassembly 110 may be utilized for re-entering the true lumen of a vessel after subintimally bypassing an occlusion in a blood vessel such as a chronic total occlusion (CTO) of an artery. FIGS. 5 and 5A illustrate an occlusion bypassing apparatus 100 that includes bendable subassembly 110 slidably disposed within an outer shaft component 102 having a balloon 112 for stabilization or anchoring the occlusion bypassing apparatus. First or intermediate tubular component 114 of bendable subassembly 110 is slidably and rotatably disposed within outer shaft component 102, and second or inner tubular component 126 is slidably and rotatably disposed within first tubular component 114. As bendable subassembly 110 extends through outer shaft component 102, occlusion bypassing apparatus 100 and bendable subassembly 110 extend in the same longitudinal direction or along the same longitudinal axis L_{A}. Occlusion bypassing apparatus 100 may also be considered to include a needle component, such as a needle 848, which is configured to be slidably disposed within lumen 132 of second tubular component 126 and removable therefrom as described in more detail below with reference to FIGS. 8 and 9. As needle 848 is a removable component, it is not shown in the configuration of FIGS. 5 and 7.

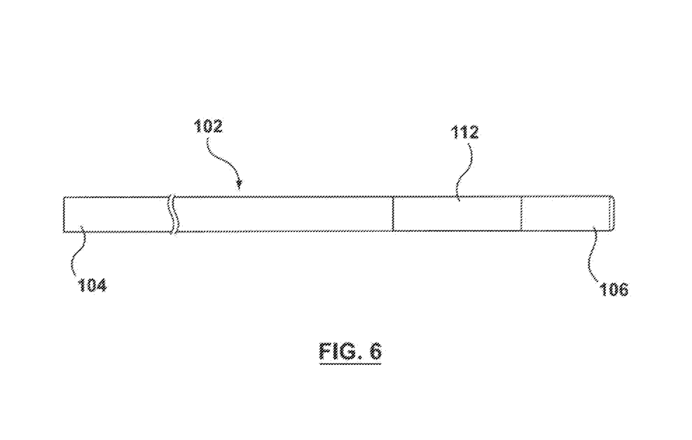

With additional reference to FIG. 6 which shows outer shaft 102 separately from occlusion bypassing apparatus 100, outer shaft component 102 is an elongate tubular or cylindrical element defining a lumen 108 that extends from a proximal end 104 to distal end 106 thereof and has balloon 112 mounted on a distal portion thereof. In an embodiment, the outer shaft component may be sized to be used with a 5F introducer sheath with lumen 108 being sized to accommodate a guidewire having an outer diameter of 0.035 inch. Proximal end 104 of outer shaft component 102 extends out of the patient and is coupled to a hub 152. An inflation shaft or tube 140 defining an inflation lumen 142 extends through lumen 108 of outer shaft component 102 to allow inflation fluid received through Luer fitting 154 of hub 152 to be delivered to balloon 112. In another embodiment hereof (not shown), the outer shaft component may include an inflation shaft or tube that is attached to extend along an outer surface of the outer shaft component to allow inflation fluid received through Luer fitting 154 of first hub 152 to be delivered to balloon 112. In accordance with an embodiment hereof, the combined structures of outer shaft component 102, balloon 112, and hub 152 as described herein may be considered to comprise a balloon catheter. It would also be understood by one of ordinary skill in the art of balloon catheter design that hub 152 includes a proximal port 156 with a hemostatic valve to accommodate insertion of other components of occlusion bypassing apparatus 100 into outer shaft component 102, and that Luer fitting 154, or some other type of fitting, may be connected to a source of inflation fluid (not shown) and may be of another construction or configuration without departing from the scope of the present invention. Other types of construction are also suitable for outer shaft component 102, such as, without limitation thereto, a catheter shaft having a central lumen and an inflation lumen formed by multi-lumen profile extrusion. When inflated, balloon 112 anchors occlusion bypassing apparatus 100 within the anatomy, more particularly within the subintimal space of the vessel wall when utilized in the treatment of a CTO, so as to provide stability thereto.

Outer shaft component 102 may be formed of polymeric materials, non-exhaustive examples of which include polyethylene terephthalate (PET), polypropylene, polyethylene, polyether block amide copolymer (PEBA), polyamide, fluoropolymers, and/or combinations thereof, either laminated, blended or co-extruded. Optionally, outer shaft component 102 or some portion thereof may be formed as a composite having a reinforcement layer incorporated within a polymeric body in order to enhance strength and/or flexibility. Suitable reinforcement layers include braiding, wire mesh layers, embedded axial wires, embedded helical or circumferential wires, hypotubes, and the like. In one embodiment, for example, at least a proximal portion of outer shaft component 102 may be formed from a reinforced polymeric tube. In accordance with an embodiment hereof, balloon 112 may radially inflate uniformly so as to have a symmetric expanded configuration about the longitudinal axis L_{A} of occlusion bypassing apparatus 100 (shown in FIG. 8). In accordance with another embodiment hereof, as described in co-pending U.S. application Ser. No. 13/952,973, balloon 112 may have an asymmetric expanded configuration relative to the longitudinal axis L_{A} of occlusion bypassing apparatus 100 (not shown) and/or may include a dual balloon arrangement that expand in opposite directions to each other (not shown) for anchoring and stabilizing the apparatus within the subintimal space.

FIG. 7 shows the distal portion of occlusion bypassing apparatus 100 with bendable subassembly 110 in the substantially straightened or delivery configuration. Notably, in the substantially straightened configuration with distal portions 122, 134 of first and second tubular components 114, 126 counterbalanced and straightened as described above with respect to FIG. 1, occlusion bypassing apparatus 100 is trackable over a guidewire 744. Guidewire 744 is an elongate substantially straight tubular or cylindrical element that is configured to be slidably disposed within lumen 132 of second tubular component 126 and removable therefrom. Lumen 132 of second tubular component 126 may be sized to slidingly receive a guidewire having a relatively small outer diameter equal to or less than 0.018 inch. A distal end 746 of guidewire 744 is shown distally extending from straightened distal portions 122, 134 of first and second tubular components 114, 126, respectively, in FIG. 7. Although occlusion bypassing apparatus 100 is shown with a guidewire extending through bendable subassembly 110 when bendable subassembly 110 is in the straightened configuration, other elongated components such as but not limited to a needle component may extend through bendable subassembly 110 in the straightened configuration.

FIG. 8 shows the distal portion of occlusion bypassing apparatus 100 with bendable subassembly 110 in the curved configuration. In the curved configuration with distal portions 122, 134 of first and second tubular components 114, 126 bent or angled, needle 848 may be advanced through bendable subassembly 110. Needle 848, which is shown removed from occlusion bypassing apparatus 100 in FIG. 9, is an elongate substantially straight tubular or cylindrical element that is configured to be slidably disposed within lumen 132 of second tubular component 126 and removable therefrom. Suitable materials for needle 848 include but are not limited to nitinol, stainless steel, or relatively hard polymeric materials such as polyetheretherketone (PEEK). When positioned through second tubular component 126, needle 848 has a proximal end 949 that proximally extends from proximal port 156 of hub 152 to be accessible by a clinician and a distal end or tip 850 configured to pierce or penetrate through a wall of a vessel. When inserted through bendable subassembly 110 in the curved or bent configuration, a distal portion or segment of needle 848 is bent by aligned elbows 124, 136 of first and second tubular components 114, 126 such that a distal segment of the needle component extends at substantially the same acute angle θ with respect to the longitudinal axis L_{A} of the apparatus as distal portions 122, 134 of the first and second tubular components. Distal tip 850 of needle 848 is shown distally extending or protruding from bent distal portions 122, 134 of first and second tubular components 114, 126, respectively, in FIG. 8. When needle 848 is distally advanced or extended as shown in FIG. 8, distal tip 850 may be used to penetrate through the vessel wall and re-enter a true lumen of a vessel as described herein. Although shown inflated or expanded in FIG. 8, balloon 112 of outer shaft component 102 may be expanded or inflated to anchor bendable subassembly 110 within a subintimal tract either before or after the distal advancement of needle 848. Although occlusion bypassing apparatus 100 is shown with a needle extending through bendable subassembly 110 when bendable subassembly 110 is in the curved configuration, in alternative to the needle other elongated components such as but not limited to a guidewire may extend through and be bent via bendable subassembly 110 in the curved configuration for re-entering the true lumen downstream of the occlusion.

In an embodiment hereof, as shown in the cross-sectional view of FIG. 9A, needle 848 may be a hypotube that defines a lumen 938 there-through from proximal end 949 to distal end 850 of the needle. Lumen 938 of needle 848 is sized to accommodate a guidewire having a relatively small outer diameter equal to or less than 0.014 inch such that occlusion bypassing apparatus 100 has a low profile. As will be described in more detail herein, such a relatively small guidewire may be inserted through needle 848 and into the true lumen of a vessel after needle 848 is utilized to gain access into the true lumen.

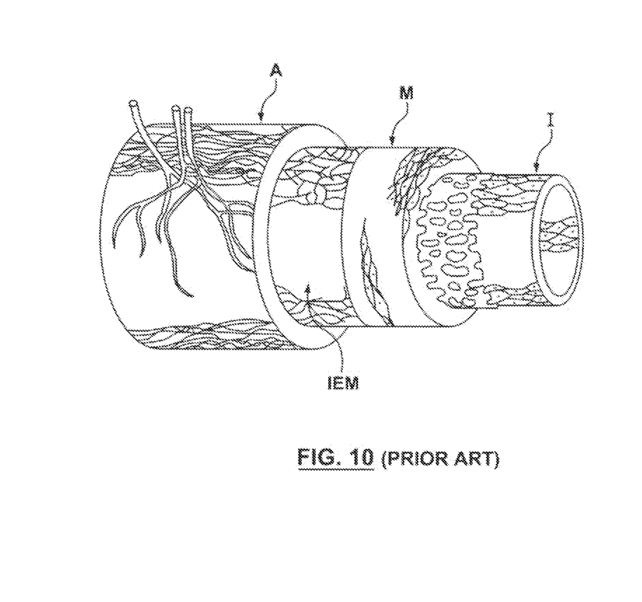

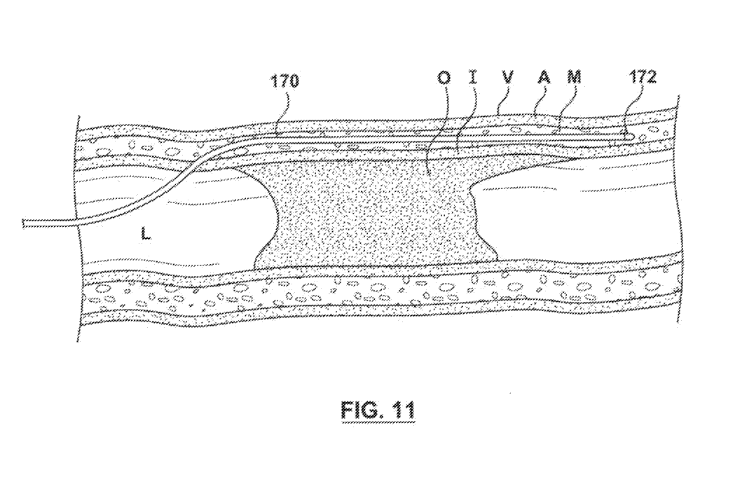

FIG. 10 is a sectional view of the anatomy of an artery wall, which for purposes of this description is shown to consist essentially of three layers, the tunica intima I (“intima”), tunica media M (“media”) which is the thickest layer of the wall, and the tunica adventitia A (“adventitia”). In some arteries an internal elastic membrane IEM is disposed between the media M and adventitia A. The adventitia A is made of collagen, vasa vasorum and nerve cells, the media M is made of smooth muscle cells, and the intima I is made up of a single layer of endothelial cells that provide a nonthrombogenic surface for flowing blood. An occlusion bypassing apparatus in accordance with embodiments hereof is used as part of a system for creating a subintimal reentry conduit within a wall of a blood vessel V to allow blood flow around an occlusion. FIGS. 11-21 illustrate an exemplary method of using the above-described occlusion bypassing apparatus 100 to bypass a chronic total occlusion (CTO) according to an embodiment hereof, but it would be understood by one of ordinary skill in the art that the depicted method may be adapted to be performed by other occlusion bypassing apparatus disclosed herein. Although described in relation to bypassing a CTO, it should be understood that the methods and apparatus described herein may be used for bypassing any tight stenoses in arteries or other anatomical conduits and are not limited to total occlusions.

As shown in FIG. 11, in accordance with techniques known in the field of interventional cardiology and/or interventional radiology, a first guidewire 170 having a distal end 172 is transluminally advanced through the vasculature to a position upstream or proximal of a treatment site, which in this instance is shown as occlusion O within a lumen L of blood vessel V. Guidewire 170 pierces the intima I and is advanced distally to create a subintimal tract by locally dissecting or delaminating intima I from media M or by burrowing through media M. Guidewire 170 has a relatively larger outer diameter such as between 0.032-0.040 inches in order to have sufficient column strength to gain access to the subintimal space of vessel V. In order to pierce the intima I, a clinician may manipulate distal end 172 of guidewire 170 by prolapsing or bending-over the distal end of guidewire 170 (not shown) and thereafter may use the stiffer arc or loop of the prolapsed distal end to pierce into the intima I to advance guidewire 170 there through. The piercing of the intima I is aided by the fact that typically blood vessel V is diseased, which in some instances makes the intima I prone to piercing. Guidewire 170 is distally advanced within the subintimal tract from a near or proximal side of occlusion O to a position where distal end 172 thereof is positioned in the subintimal tract on a far or distal side of occlusion O.

Alternatively, another device other than guidewire 170 may be initially used to create the subintimal tract. Those of ordinary skill in the art will appreciate and understand the types of alternative devices that may be used in this step including an apparatus known as an “olive”, a laser wire, an elongate radiofrequency electrode, a microcatheter, a guiding catheter, or any other device suitable for boring or advancing through the vessel tissue. If an alternative device is used instead of guidewire 170 to form the subintimal tract, such alternative device may be removed and replaced with guidewire 170 or a smaller diameter guidewire after the subintimal tract has been formed.

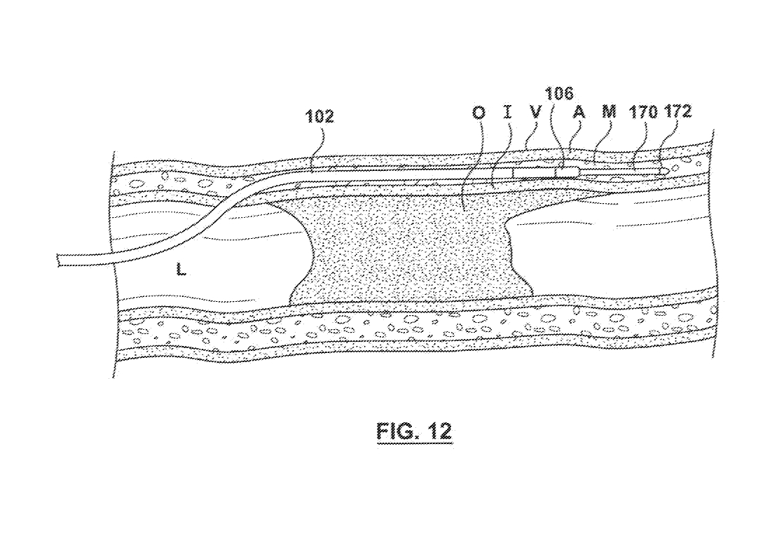

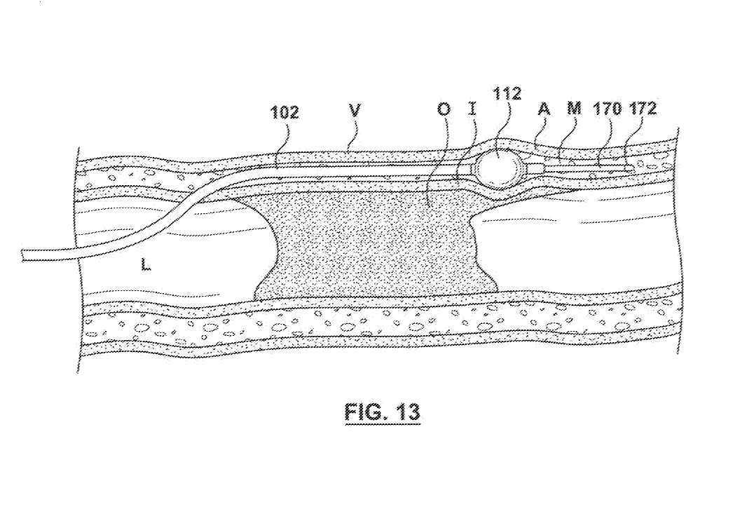

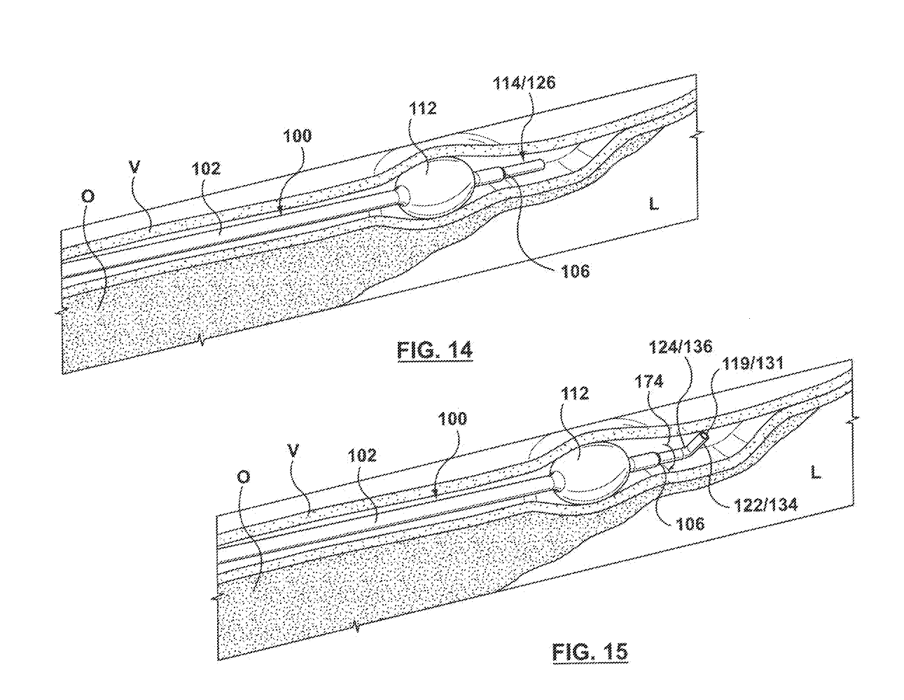

After the subintimal tract is formed, outer shaft component 102 of occlusion bypassing apparatus 100 is tracked over guidewire 170 and advanced until distal end 106 of outer shaft component 102 is disposed at the far end of occlusion O as shown in FIG. 12. Once outer shaft component 102 is positioned as desired, balloon 112 may be inflated as shown in FIG. 13, thus anchoring outer shaft component 102, and in case occlusion bypassing apparatus 100, in the subintimal tract. Guidewire 170 may then be proximally refracted and removed, and the bendable subassembly of first tubular component 114 with second tubular component 126 disposed therein are concurrently loaded into and advanced through outer shaft component 102. During advancement or loading of the bendable subassembly of first and second tubular components 114, 126 through outer shaft component 102, distal portions 122, 134 of first and second tubular components 114, 126, respectively, are substantially straightened or counterbalanced as described above with respect to FIG. 1. First and second tubular components 114, 126 are advanced within outer shaft component 102 until distal portions 122, 134 thereof distally extend from distal end 106 of outer shaft component 102, as shown in FIG. 14. Although inflation of balloon 112 is described as occurring prior to insertion of first and second tubular components 114, 126, in another embodiment hereof (not shown) inflation of balloon 112 may not occur until after positioning of the first and second tubular components within outer shaft component 102 so long as balloon inflation occurs prior to bending and/or rotation of distal portions 122, 134 of first and second tubular components 114, 126 as described herein.

After occlusion bypassing apparatus 100 is positioned adjacent to the far or downstream end of occlusion O as desired with balloon 112 inflated and distal portions 122, 134 of first and second tubular components 114, 126 distally extending from distal end 106 of outer shaft component 102, one of first and second tubular components 114, 126 is rotated relative to the other to transform distal portions 122, 134 thereof into the curved or bent configuration as described above with respect to FIG. 2. When rotated such that elbows 124, 136 are aligned, distal portions 122, 134 of first and second tubular components 114, 126 resume their shape memory geometry by their own internal restoring forces and concurrently bend, curve, or bow away from the longitudinal axis of occlusion bypassing apparatus 100 as shown in FIG. 15 to orient distal openings 119, 131 of first and second tubular components 114, 126 towards true lumen L of vessel V. If present, marker(s) 117 of one or both of first tubular component 114 and second tubular component 126 assist the user in tracking the degree of rotation of the tubular components to indicate when first and second tubular components 114, 126 have been rotated approximately 180° with respect to each other, thereby indicating when the straight configuration has been achieved.

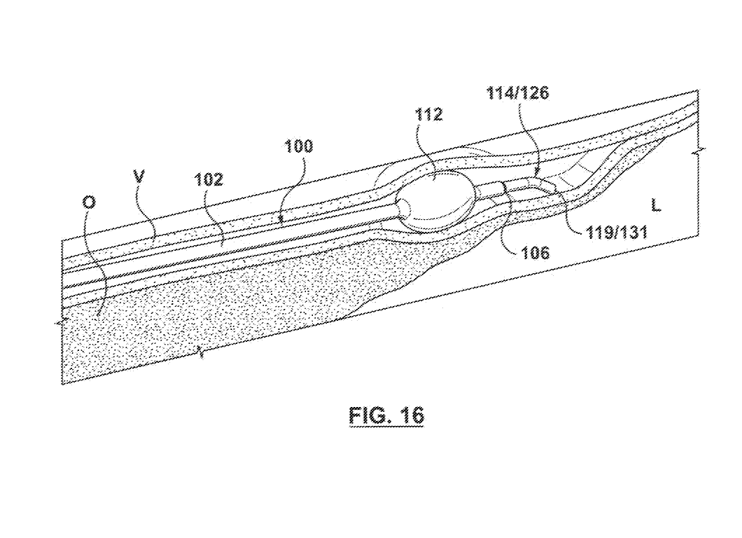

If distal openings 119, 131 of first and second tubular components 114, 126 are not oriented or pointed towards true lumen L of vessel V, the bendable subassembly of first and second tubular components 114, 126 may be jointly or collectively rotated or turned by a physician as an ensemble as shown by a directional arrow 174 in FIG. 15 to make any necessary adjustment of the rotational position or orientation of the bendable subassembly within the subintimal tract to ensure that distal tip 850 of needle 848, which is successively loaded into subassembly 110, will be deployed into a specific radial location, i.e. into the intima I, on the vessel wall in order to access the true lumen downstream of the occlusion. In an embodiment, a removable locking device or wire torquer may be utilized at the proximal ends of first and second tubular components 114, 126 during simultaneous rotation thereof, wherein the locking device or wire torquer may then be removed when distal openings 119, 131 of first and second tubular components 114, 126 are oriented as desired towards true lumen L of vessel V. First and/or second tubular components 114, 126 may include a radiopaque marker (not shown) at their respective distal ends in order to assist in orienting the bendable subassembly towards the true lumen. In another embodiment hereof, a portion of first and/or second tubular components 114, 126 may be formed from a radiopaque material to assist in orienting the bendable subassembly towards the true lumen. The correct rotational position or orientation of first and second tubular components 114, 126 is shown in FIG. 16, with distal openings 119, 131 thereof oriented toward the true lumen of the vessel.

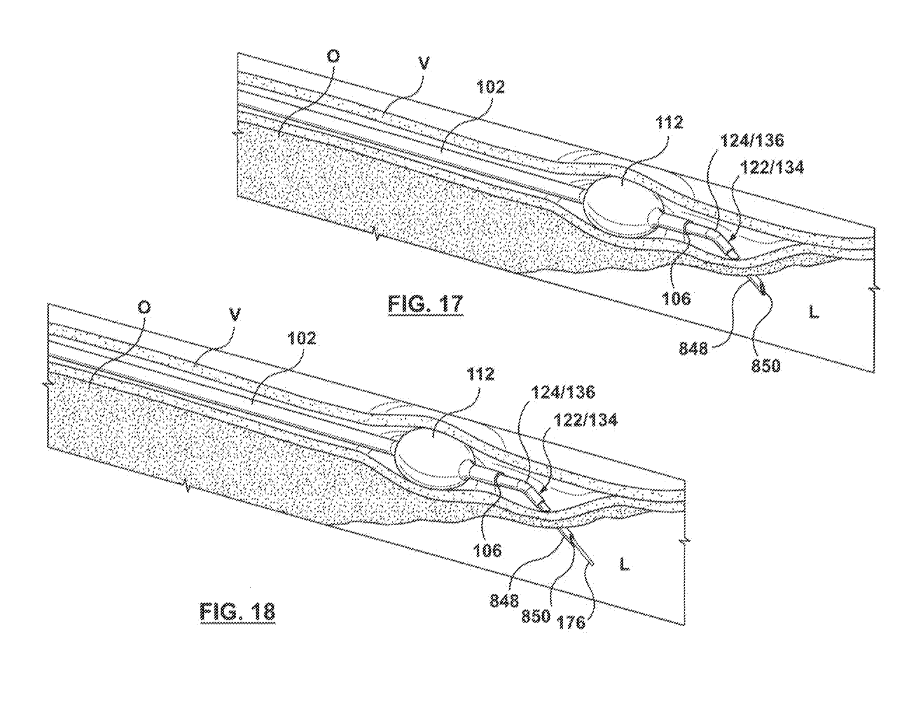

Once distal openings 119, 131 of first and second tubular components 114, 126 are oriented towards the vessel true lumen as desired, needle 848 is introduced into proximal end 128 of second tubular component 126 and distally advanced through second tubular component 126 until distal tip 850 of needle 848 extends from or protrudes out of distal opening 131 of second tubular component 126 and penetrates the intima to gain access to the true lumen of the vessel distal to, i.e., downstream of, the CTO as shown in FIG. 17. When inserted through bendable subassembly 110 in the curved or bent configuration, a distal portion or segment of needle 848 is bent by elbows 124, 136 of first and second tubular components 114, 126 such that a distal segment of the needle component extends at substantially the same acute angle with respect to the longitudinal axis of the apparatus as distal portions 122, 134 of the first and second tubular components. In another embodiment hereof (not shown), needle 848 may be introduced into proximal end 128 of second tubular component 126 prior to rotating first and/or second tubular components 114, 126 to bend distal portions 122, 134 thereof as described above with respect to FIGS. 15-16. More particularly, needle 848 may be introduced into second tubular component 126 and disposed or housed only within proximal portion 114, 126 of subassembly 110 while rotating first and/or second tubular components 114, 126 to bend distal portions 122, 134 thereof. Then, after subassembly 110 is in the bent configuration, needle 848 may be further distally advanced until distal tip 850 of needle 848 extends from or protrudes out of distal opening 131 of second tubular component 126. When the needle is further distally advanced, the distal portion or segment of needle 848 is bent by elbows 124, 136 of first and second tubular components 114, 126 as described above with respect to FIG. 17.

In addition, prior to advancing the distal portion of needle 848 out from first and second tubular components 114, 126, a small amount of longitudinal or axial movement between second tubular component 126 and first tubular component 114 may provide better support of needle 848 when the needle is extended out of the bendable subassembly. More particularly, it may be desirable to slightly distally advance, i.e., between 1-3 millimeters, second tubular component 126 with respect to first tubular component 114 prior to distally advancing needle 848 out of second tubular component 126. This small amount of longitudinal or axial movement between the tubular components may slightly offset or wedge the second tubular component within the first tubular component and thus provide improved or better support to needle 848.

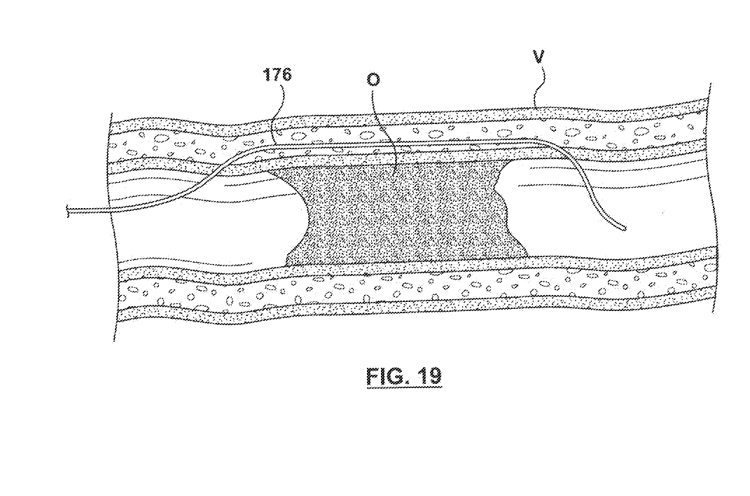

After the puncture has occurred and the true lumen has been accessed, a second guidewire 176 may be advanced through lumen 938 of needle 848 and into the true lumen L of vessel V as shown in FIG. 18. Guidewire 176 has a relatively smaller outer diameter such as 0.018 inches or less in order to minimize the size of needle 848 and subsequently minimize the size of occlusion bypassing apparatus 100. Optionally, occlusion bypassing apparatus 100 may be removed and guidewire 176 may be left in place as shown in FIG. 19, such that guidewire 176 extends in true lumen L proximal to the CTO, through the subintimal tract, and back into true lumen L distal to the CTO to enable the CTO to be successfully crossed via the subintimal conduit thus created.

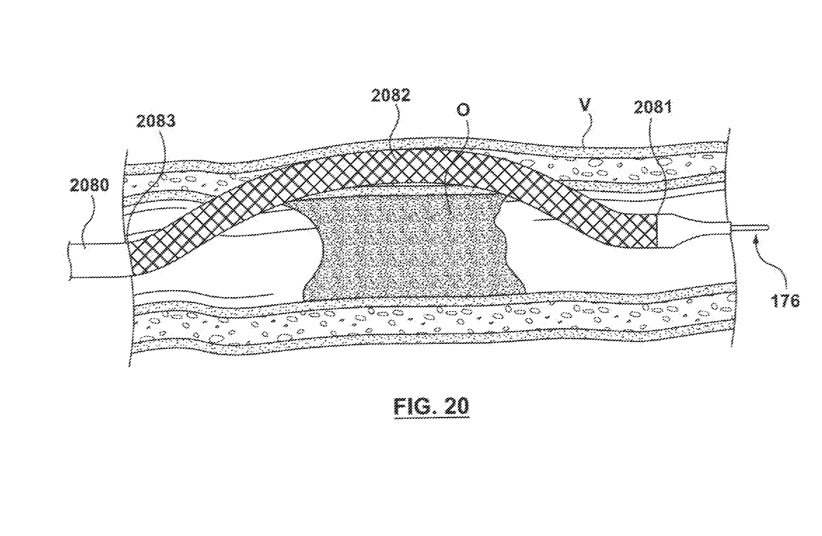

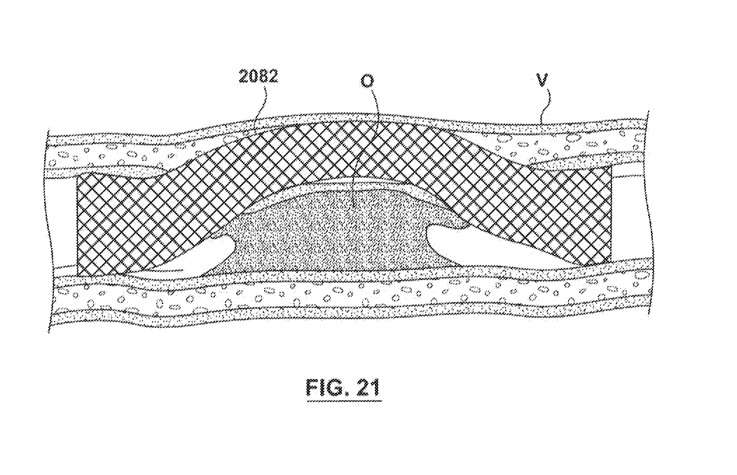

Optionally, a covered or uncovered stent may be delivered over guidewire 176 and implanted within the subintimal tract to facilitate flow from the lumen of the vessel upstream of the CTO, through the subintimal tract and back into the lumen of the vessel downstream of the CTO. For example, FIG. 20 shows a distal end of a catheter 2080 having a stent 2082 mounted thereon being advanced over guidewire 176 to a position where a distal end 2081 of the radially collapsed stent 2082 is in true lumen L of vessel V downstream of chronic total occlusion CTO, a proximal end 2083 of stent 2082 is in true lumen L of vessel V upstream of chronic total occlusion CTO, and a tubular body of stent 2082 extends through the subintimal tract. Stent 2082 is then deployed by either self-expansion or balloon inflation within the subintimal reentry conduit to dilate the subintimal tract and compress the adjacent chronic total occlusion CTO. Stent 2082 provides a scaffold which maintains the subintimal tract in an open condition capable of carrying blood downstream of chronic total occlusion CTO. Thereafter, guidewire 176 and catheter 2080 may be removed from the patient, leaving stent 2082 in an expanded configuration and creating a radially supported, subintimal blood flow channel around chronic total occlusion CTO as seen in FIG. 21. In some cases, it may be desirable to enlarge the diameter of the subintimal tract before advancing stent catheter 2080 into and through it. Such enlargement of the subintimal tract may be accomplished by passing a balloon dilatation catheter over guidewire 176 and inflating the balloon to dilate the tract, or may be any other suitable tract enlarging, dilating or de-bulking instrument that may be passed over guidewire 176.

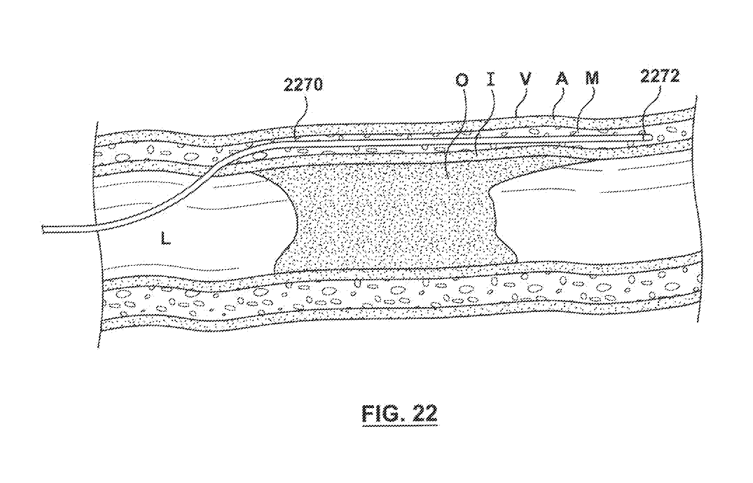

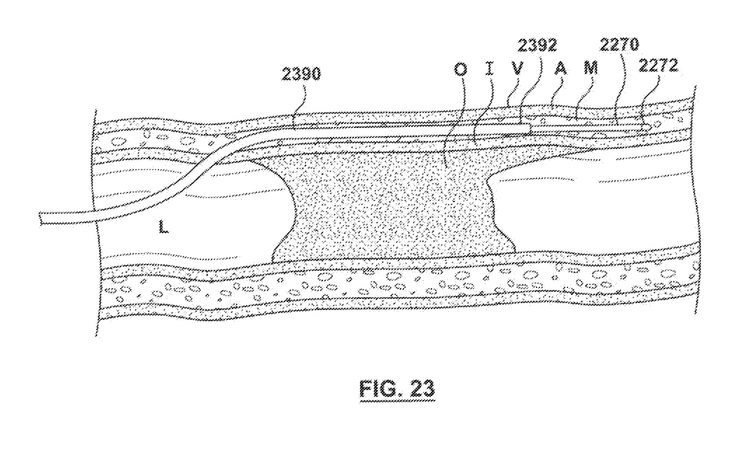

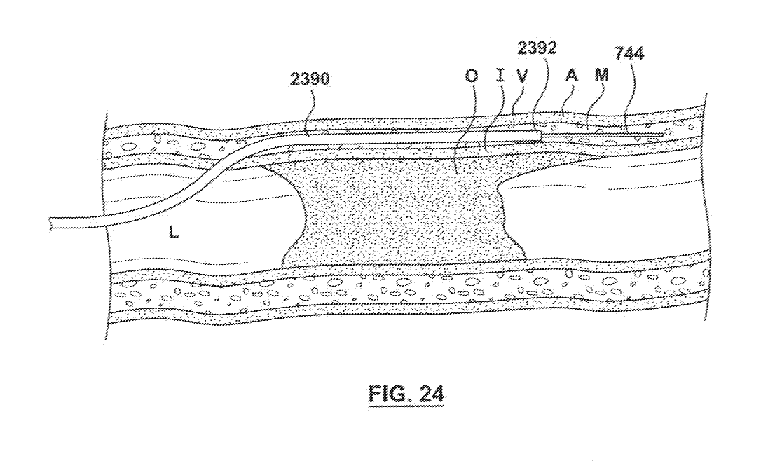

FIGS. 22-27 illustrate an alternative method of forming a subintimal tract and positioning occlusion bypassing apparatus 100 adjacent to the distal or downstream end of occlusion O. With reference to FIG. 22, a first guidewire 2270 having a distal end 2272 is transluminally advanced through the vasculature to a position proximal or upstream of a total occlusion O within a lumen L of blood vessel V. Similar to guidewire 170, guidewire 2270 has a relatively larger outer diameter in order to have sufficient column strength to gain access to the subintimal space of vessel V and guidewire 2270 is utilized to pierce the intima I and create a subintimal tract between the intima I and the media M. A guide catheter 2390 is then tracked over guidewire 2270 and advanced such that a distal end 2392 thereof is adjacent to the distal or downstream end of occlusion O as shown in FIG. 23. Guidewire 2270 may then be proximally retracted and removed, and relatively smaller second guidewire 744 may be loaded into and advanced through guide catheter 2390 as shown in FIG. 24. As described above with respect to FIG. 7, guidewire 744 has a relatively smaller outer diameter such as 0.014 inches.

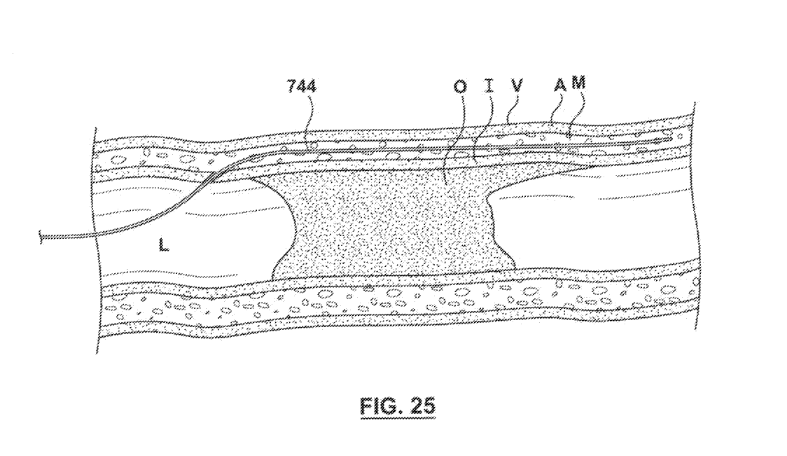

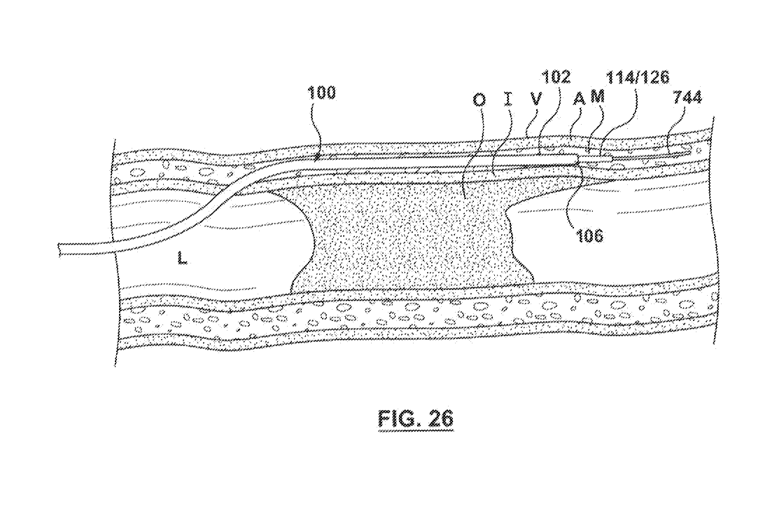

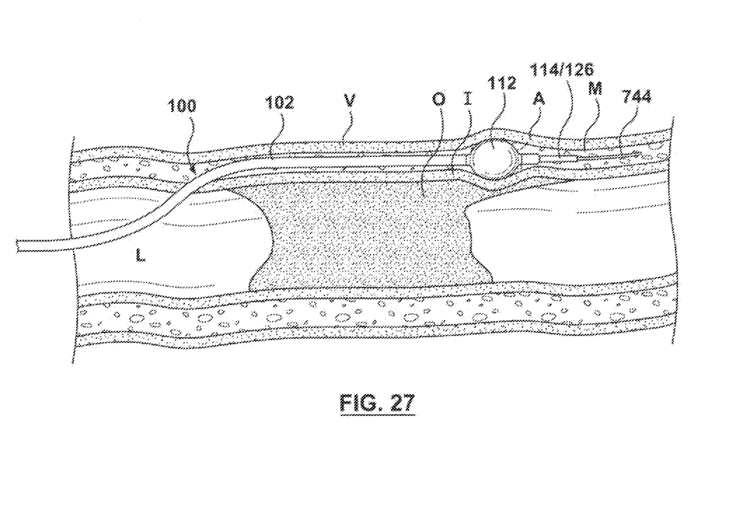

After second guidewire 744 is in place as desired, guide catheter 2390 may be proximally retracted and removed as shown in FIG. 25, leaving only second guidewire 744 extending into the subintimal tract. At this point, occlusion bypassing apparatus 100 may be tracked over second guidewire 744 and advanced such that a distal end 106 of outer shaft 102 is adjacent to the distal end of occlusion O as shown in FIG. 26. In this embodiment, outer shaft component 102 and the bendable subassembly 110 of first tubular component 114 and second tubular component 126 are concurrently advanced as an ensemble over second guidewire 744 rather than advancing the outer shaft component prior to the first and second tubular components as described in the prior embodiment. Stated another way, bendable subassembly 110 of first and second tubular components 114, 126 is loaded into outer shaft component 102 prior to the step of advancing occlusion bypassing apparatus 100 over guidewire 744. As shown in FIG. 26, during distal advancement of occlusion bypassing apparatus 100, distal portions 122, 134 of first and second tubular components 114, 126 are in the substantially straightened or counterbalanced configuration. Once occlusion bypassing apparatus 100 is positioned as desired, balloon 112 may be inflated as shown in FIG. 27 to anchor outer shaft component 102 in the subintimal tract. Guidewire 744 may be proximally retracted and removed, leaving only occlusion bypassing apparatus 100 extending through the subintimal tract.

Once occlusion bypassing apparatus 100 is positioned adjacent to the distal end of occlusion O as desired with balloon 112 inflated, the remaining steps to create a subintimal conduit that bypasses the occlusion O are the same as described with respect to FIGS. 15-21. More particularly, distal portions 122, 134 of first and second tubular components 114, 126 may be bent via rotation of one of the first or second tubular components and the bendable subassembly of the first and second tubular components may be rotated as described above with respect to FIGS. 15-16. Distal tip 850 of needle 848 is then distally advanced to penetrate through the intima and thereafter pass into the true lumen of the vessel as described with respect to FIG. 17, and a guidewire may be advanced through needle 848 into the true lumen of the vessel as described with respect to FIG. 18. Optionally, a stent may be delivered and implanted within the subintimal tract to facilitate flow from the lumen of the vessel proximal of the CTO, through the subintimal tract and back into the lumen of the vessel distal of the CTO as described with respect to FIGS. 19-21.

In another embodiment hereof (not shown), rather than removing guidewire 744 as described with respect to FIG. 26, guidewire 744 may be left extending through occlusion bypassing apparatus 100 for the remaining steps of the procedure. If guidewire 744 is left in place, it is slightly retracted when bendable subassembly is transformed to the curved configuration so as to not interfere with distal portions 122, 134 of first and second tubular components 114, 126 during bending thereof. After bending has occurred, guidewire 744 is distally advanced or repositioned through distal portions 122, 134 of first and second tubular components 114, 126 and needle 848 is then distally advanced over guidewire 744 until distal tip 850 of the needle penetrates through the intima and into the true lumen of the vessel.

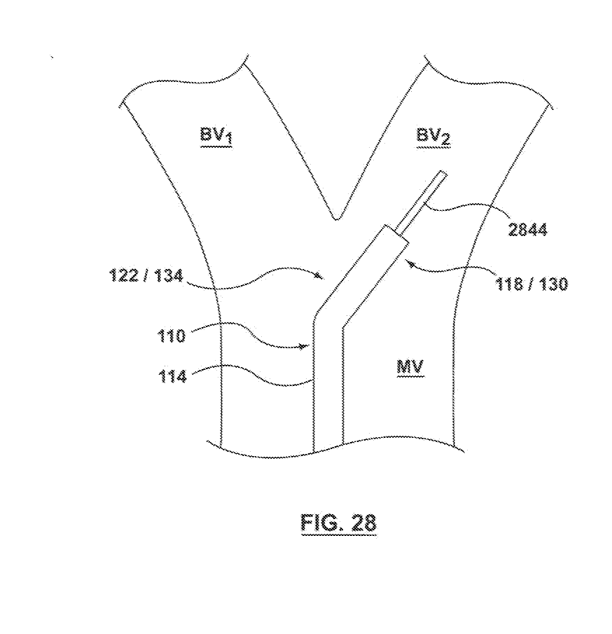

Although shown and described in use with a balloon catheter, e.g., outer shaft component 102 having balloon 112, occlusion bypassing apparatus 100 does not necessarily require the presence of balloon 112 and bendable subassembly 110 may be utilized with other types of catheters suitable for crossing an occlusion. Further, in addition to being useful for crossing a CTO as described above, bendable subassembly 110 of first and second tubular components 114, 126 may be useful in other applications. More particularly, first and second tubular components 114, 126 may be utilized in any application in which it is desirable to orient or position a distal end of the apparatus in a direction different from that of the longitudinal axis of the apparatus, such as during navigation within a bifurcation or through tortuous anatomy. For example, referring to FIG. 28, bendable subassembly 110 is shown within the vasculature at a bifurcation having a main vessel MV, a first branch vessel BV₁, and a second branch vessel BV₂. When distal portions 122, 134 of first and second tubular components 114, 126 are configured in the curved or bent configuration as described above, distal ends 118, 130 of first and second tubular components 114, 126 are directed towards second branch vessel BV₂. A guidewire 2844 is shown inserted through bendable subassembly 110 and extending into second branch vessel BV₂. If it is desired to direct distal ends 118, 130 of first and second tubular components 114, 126 towards first branch vessel BV₁, first and second tubular components 114, 126 may be jointly or collectively rotated in situ such that distal portions 122, 134 point towards first branch vessel BV₁. Thus, the bending direction of distal ends 118, 130 of first and second tubular components 114, 126 may be selectively decided in situ by the operator in order to direct the distal end of the apparatus towards a particular branch of the bifurcation, i.e., first branch vessel BV₁ and a second branch vessel BV₂. Guidewire 2844 inserted there-through is thus directed towards a specific endovascular region. Although not shown in FIG. 28, bendable subassembly 110 may be used with a variety of catheters trackable over a guidewire, such as but not limited to balloon catheters. More particularly, as previously described, the combined structures of outer shaft component 102, balloon 112, and hub 152 as described above with respect to occlusion bypassing system 100 may be considered to comprise a balloon catheter. As such, when utilized therewith in the vasculature, first and second tubular components 114, 126 may be considered a subassembly utilized for steering or navigating the balloon catheter through vasculature of a patient. Exemplary applications include accessing the carotid, iliac or renal bifurcations, in either diagnostic or therapeutic applications.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An apparatus for use within a vasculature of a patient comprising:
a first tubular component having a distal portion that is at an acute angle with respect to a longitudinal axis of the apparatus; and
a second tubular component rotatably disposed within the first tubular component and sized to be rotatable relative thereto, the second tubular component having a distal portion that is at an acute angle with respect to the longitudinal axis of the apparatus,
wherein the apparatus has a curved configuration when the distal portions of the first and second tubular components are positioned to extend at substantially the same acute angle with respect to the longitudinal axis of the apparatus, and
wherein the apparatus has a substantially straightened configuration when the distal portions of the first and second tubular components are positioned such that the curvatures of the first and second tubular components counterbalance each other.

2. The apparatus of claim 1, wherein each of the first and second tubular components is formed to have a pre-set curved form in which the distal portion is curved away from a longitudinal axis thereof at the acute angle.

3. The apparatus of claim 2, wherein rotation of the first tubular component relative to the second tubular component configures the apparatus in one of the curved and straightened configurations.

4. The apparatus of claim 3, wherein 180 degree rotation of the first tubular component relative to the second tubular component configures the apparatus in one of the curved and straightened configurations and wherein at least one of the first and second tubular components include a marker at a proximal end thereof for tracking the degree of relative rotation between the first and second tubular components.

5. The apparatus of claim 2, wherein when the apparatus is in the straightened configuration the first and second tubular components are positioned with their distal portions curved in opposite directions from each other such that the pre-set curved forms of the first and second tubular components counterbalance each other.

6. The apparatus of claim 1, wherein the second tubular component defines a lumen from a proximal end to a distal end thereof and the apparatus further comprises a needle component configured to be slidably disposed within the lumen of the second tubular component.

7. The apparatus of claim 6, wherein the needle component has a distal tip for penetrating a wall of the vessel.

8. The apparatus of claim 6, wherein when the apparatus is in the curved configuration the needle component is bent by the first and second tubular components such that a distal segment of the needle component extends at substantially the same acute angle with respect to the longitudinal axis of the apparatus as the distal portions of the first and second tubular components.

9. An apparatus for use within a vasculature of a patient comprising:
a first tubular component defining a lumen from a proximal end to a distal end thereof and having an elbow beyond which a distal portion of the first tubular component is at an acute angle with respect to a longitudinal axis of the apparatus; and
a second tubular component disposed within the lumen of the first tubular component and sized to be rotatable relative thereto, the second tubular component having an elbow beyond which a distal portion of the second tubular component is at an acute angle with respect to the longitudinal axis of the apparatus,
wherein in a first configuration of the apparatus, the elbows of the first and second tubular components are aligned with each other such that the distal portions of the first and second tubular components are bent at substantially the same acute angle with respect to the longitudinal axis of the apparatus, and
wherein in a second configuration of the apparatus, the elbows of the first and second tubular components are oriented in opposite directions from each other such that the distal portions of the first and second tubular components are straightened to substantially extend along the longitudinal axis of the apparatus.

10. The apparatus of claim 9, wherein the first and second tubular components are rotated relative to each to other to configure the apparatus in one of the first and second configurations.

11. The apparatus of claim 10, wherein the first and second tubular components are rotated approximately 180 degrees relative to each to other to configure the apparatus in one of the first and second configurations and wherein at least one of the first and second tubular components include a marker at a proximal end thereof for tracking the degree of relative rotation between the first and second tubular components.

12. The apparatus of claim 9, wherein the second tubular component defines a lumen from a proximal end to a distal end thereof and the apparatus further comprises a needle component configured to be slidably disposed within the lumen of the second tubular component, wherein the needle component has a distal tip for penetrating a wall of the vessel.

13. The apparatus of claim 12, wherein when the apparatus is in the first configuration the needle component is bent by the aligned elbows of the first and second tubular components such that a distal segment of the needle component extends at substantially the same acute angle with respect to the longitudinal axis of the apparatus as the distal portions of the first and second tubular components.

14. The apparatus of claim 9, wherein each of the first and second tubular components is formed to have a pre-set curved form in which the distal portion is curved away from a longitudinal axis thereof at the acute angle.

15. The apparatus of claim 14, wherein when the apparatus is in the second configuration the first and second tubular components are positioned with their distal portions curved in opposite directions from each other such that the pre-set curved forms of the first and second tubular components counterbalance each other.

16. A method of orienting a distal end of an apparatus in situ, the method comprising the steps of:
percutaneously advancing the apparatus through a vasculature to a target location, wherein the apparatus includes a first tubular component and a second tubular component rotatably disposed within the first tubular component, the first and second tubular components each having a distal portion that is at an acute angle with respect to the longitudinal axis of the apparatus, wherein the apparatus is in a substantially straightened configuration during the step of advancing the apparatus through a vasculature in which the distal portions of the first and second tubular components are positioned such that the curvatures of the first and second tubular components counterbalance each other; and
rotating at least one of the first tubular component and the second tubular component to configure the apparatus into a curved configuration, wherein in the curved configuration the distal portions of the first and second tubular components are positioned to extend at substantially the same acute angle with respect to the longitudinal axis of the apparatus.

17. The method of claim 16, wherein the step of rotating the first tubular component and the second tubular component relative to each other occurs within a subintimal space of a chronic total occlusion.

18. The method of claim 17, further comprising:
distally advancing a needle component through the second tubular component when the apparatus is in the curved configuration, wherein the needle component is bent by the first and second tubular components such that a distal segment of the needle component extends at substantially the same acute angle with respect to the longitudinal axis of the apparatus as the distal portions of the first and second tubular components.

19. The method of claim 16, wherein the step of rotating the first tubular component and the second tubular component relative to each other occurs at a bifurcation within a vasculature.

20. The method of claim 19, further comprising:
distally advancing a guidewire through the second tubular component when the apparatus is in the curved configuration, wherein the guidewire is bent by the first and second tubular components such that a distal segment of the guidewire extends at substantially the same acute angle with respect to the longitudinal axis of the apparatus as the distal portions of the first and second tubular components.

21. The method of claim 16, wherein the step of rotating at least one of the first tubular component and the second tubular component to configure the apparatus into a curved configuration includes rotating the first and second tubular components approximately 180 degrees relative to each to other.
